(19) United States
(12) Patent Application Publication
(10) Pub. No.: US 2010/0311779 A1
(43) Pub. Date: Dec. 9, 2010

(54) METHODS FOR PREPARING DEHYDROCAVIDINE, DEHYDROAPOCAVIDINE OR THEIR COMPOSITION, THEIR USE AND MEDICINAL COMPOSITON CONTAINING THEM

(76) Inventors: Weidong ZHANG, Shanghai (CN); Huiliang Li, Shanghai (CN); Chuan Zhang, Shanghai (CN); Runhui Liu, Shanghai (CN); Juan Su, Shanghai (CN); Xike Xu, Shanghai (CN); Hanxiong Li, Guangzhou (CN)

Correspondence Address: Muncy, Geissler, Olds & Lowe, PLLC, 4000 Legato Road, Suite 310, FAIRFAX, VA, 22033, US

(21) Appl. No.: 12/794,392

(22) Filed: Jun. 4, 2010

Related U.S. Application Data
(63) Continuation-in-part of application No. 11/806,874, filed on Jun. 5, 2007, now Pat. No. 7,732,458.
Application No. 11/806,874 is a continuation of application No. PCT/CN2005/001755, filed on Oct. 24, 2005.

(51) Int. Cl. A61K 31/4741; C07D 491/056; A61P 31/12; A61P 31/16; A61P 35/00; A61P 9/06; A61P 37/00; A61P 1/16

(52) U.S. Cl. 514/280; 546/48

(57) ABSTRACT

A method for preparing dehydrocavidine, dehydroapocavidine and their respective composition is provided. The composition is first prepared by isolating and purifying the quaternary ammonium alkaloid components from the medicinal plant “Yan Huang Lian” (Corydalis saxicola Bunting) through the processes of solvent extraction, water-phase organic extraction, crystallization and recrystallization, and then drying to obtain said composition containing dehydrocavidine and dehydroapocavine. When necessary, the composition or their crude extracts can be separated by chromatography to obtain dehydrocavidine or dehydroapocavidine. Dehydrocavidine, dehydroapocavidine or their respective composition can be used in manufacturing medicines for treating viral hepatitis, hepatic injury, influenza, AIDS, tumors or arrhythmia.

This application is a continuation-in-part, and claims priority, of from U.S. patent application Ser. No. 11/806,874 filed on Jun. 5, 2007, which is a continuation of PCT from PCT Patent Application No. PCT/CN2005/01755 filed on Oct. 24, 2005, entitled “METHODS FOR PREPARING DEHYDROCAVIDINE, DEHYDROAPOCAVIDINE OR THEIR COMPOSITION, THEIR USE AND MEDICINAL COMPOSITION CONTAINING THEM”, the entire contents of which are hereby incorporated by reference.

### FIELD OF THE INVENTION

The present invention discloses a method for preparing dehydrocavidine (FIG. 1), dehydroapocavidine and their respective composition, comprising the following steps: isolating and purifying the quaternary ammonium alkaloid components from the medicinal plant “Yan Huang Lian” (Corydalis saxicola Bunting) through the processes of solvent extraction, water-phase organic extraction, crystallization and recrystallization, and then drying to obtain said composition containing dehydrocavidine and dehydroapocavine. When necessary, said composition or their crude extracts obtained from said steps can be separated by chromatography to obtain dehydrocavidine or dehydroapocavidine. Dehydrocavidine, dehydroapocavidine or their respective composition can be used in manufacturing medicines for treating viral hepatitis, hepatic injury, influenza, AIDS, tumors or arrhythmia.

The present invention relates to the fields of medicine and pharmacology, in particular, to a method of extracting dehydrocavidine, dehydroapocavidine, and their respective composition from a medicinal plant of Yan Huang Lian (Corydalis saxicola Bunting), and their pharmaceutical use.

### BACKGROUND OF THE INVENTION

Hepatitis is one of the most harmful infectious diseases in the world, and is also related closely to the onset of liver cancer. According to statistical data from the World Health Organization (WHO), presently there are 350 million chronic hepatitis sufferers or asymptomatic patients infected by hepatitis viruses all over the world. All of these chronic hepatitis patients are at high risk of developing hepatocirrhosis and liver cancer. Over one million people die of diseases related to hepatitis, hepatocirrhosis and liver cancer each year. The incidence of hepatitis cases in China is high, and data shows that 90 percent of liver cancer patients were infected by the hepatitis viruses. Currently liver cancer is the second-most fatal tumor disease. To prevent and treat such a common, frequently occurring and refractory disease, researchers inside and outside of China have been focusing on and conducting clinical and pharmacological studies in liver protection and detoxification. Most countries treat chronic hepatitis B with a-interferon whose main effect is immunoregulation. Presently there are no specific drugs that can effectively treat hepatitis B and C caused by their respective viruses. The drugs against hepatitis B viruses are mostly the anti-HIV reverse transcriptase inhibitors and anti-herpes viruses DNA polymerase inhibitors. These two types of virus enzyme inhibitors are the target of anti-hepatitis B virus. The drugs used to fight hepatitis C viruses are mostly the broad-spectrum anti-virus drugs or RNA-virus inhibitors, and the immunoregulators having the activity of anti-viruses. However, the general problem with drugs currently available to use against hepatitis is that they are subject to drug-resistance.

Yan Huang Lian (Corydalis saxicola Bunting [Corydalis thalictrifolia Franch. Non Jameson ex Regel]), is a whole plant belonging to the family of papaveraceae. It is also known as Yan Hu (Guizhou), Yan Lian (Sichuan, Yunnan), Ju Hua Huang or Tu Huang Lian (Guangxi). Native residents in Guangxi use the roots of the plant as a pain killer, for detumescence, for drawing out pus and for treatment of scabies and swelling. Its current clinical application includes the use of its alkaloid extracts to treat hepatitis and hepatocirrhosis (Editorial Board of China Herbal. 1999. State Administration of Traditional Chinese Medicine. China Herbal, Vol. 3. Shanghai Science and Technology Press: Shanghai; 638-640).

In 1982, Chongyang Chen et al (Chen C Y, 1982. Pharmacological study of dehydrocavidine, the major constituent in Yan Huang-lian (Corydalis, saxicola Bunting). Trad Chin Med 7: 31-34.) investigated the pharmacological activity of dehydrocavidine which is the main constituent in Corydalis saxicola Bunting, and the results indicated that dehydrocavidine had sedative effects on the central nervous system; antispasm effects on the smooth muscles of the intestines; antibacterial effect in vitro; no effects on the blood sugar levels in normal mice; and effect on increasing the production of glycogen in vivo. In 1984, Qili Ye (Ye Q L. 1984. Anti-bacterial effect of dehydrocavidine from Corydalis saxicola. Gaungxi Zhongyiyao 3: 48-49.) investigated the anti-bacteria activity of dehydrocavidine in vitro, and the experiment proved that the dehydrocavidine had certain inhibitory effect on gram-positive bacterium. In 1996, Peishan Xie, et al. (Xie Peishan et al, “Screening tests of Chinese traditional medicines or herbal medicines in antitumor activity.” Shizhen Journal of Traditional Chinese Medicine Research, Vol. 7, no. 1, 1996, pages 19-20), reported that their anti-tumor experiment with this herb proved that the total alkali of Corydalis saxicola Bunting had a 30 percent inhibition rate on S180 carunclesarcoma at the dose of 1.6 mg/kg. In the past 10 years, some studies have indicated that the total alkali of Corydalis saxicola Bunting had an enhanced effect on the immune functions of mice, and certain inhibitory effects on the metabolism of DA and 5-HT in the rat's brain.

Yan Huang Lian (Corydalis saxicola Bunting) is clinically used as a supplementary therapeutic treatment of hepatitis. A study conducted by Zhongxuan Ren (Zhongxuan Ren, the efficacy analysis of 33 hepatitis cases treated with Yan Huang Lian (Corydalis saxicola Bunting), Clinical Focus, 18 (2): 94-95, 2003) showed that the injection of Yan Huang Lian (Corydalis saxicola Bunting Injecta) could effectively improve the clinical symptom of acute and chronic hepatitis. The Yan Huang Lian Injection combined with Shengmai injection have a distinct curative effect on hepatocirrhosis. Yan Huang Lian Injection combined with Danshen injection can effectively improve liver function, and relieve and inhibit liver fibrosis.

Although Corydalis saxicola Bunting has good clinical effects, the active constituents of this plant are still unclear and there are no practically feasible quality standards because of the lack of in-depth research on chemical constituents and the lack of sufficient screening of pharmacological activities.

### SUMMARY OF THE INVENTION

To overcome the shortcoming of the current art and solve the technical problems mentioned above, the present invention is to extract potent active constituents from Yan Huang Lian (Corydalis saxicola Bunting), a Chinese traditional medicinal plant, and is to screen out from these natural products the lead compounds with anti-hepatitis activities, and then screen out more active single compound from a series of derivative compounds through modification of chemical structure and synthesis of the lead compounds, and by combining with the study on relationship between structure and efficacy of anti-hepatitis B virus activities, the invention is to eventually lead to the discovery of drugs with promising clinical applications.

A plentiful quaternary ammonium alkali species and tertiary ammonium alkali species alkaloids that exist in Yan Huang Lian (Corydalis saxicola Bunting) are discovered by a systematic phytochemical separation, purification and structural identification of chemical constituents in this plant. Further screening of the pharmacological activity has proved that there are mainly dehydrocavidine and dehydroapocavidine in the quaternary ammonium alkali species and they are the active constituents against hepatitis, hepatitis B virus, tumor and arrhythmia.

FIG. 1 and FIG. 2 show the structural formula of dehydrocavidine and dehydroapocavidine. According to the chemical properties and solubility of dehydrocavidine and dehydroapocavidine, dehydrocavidine-dehydroapocavidine composition and their respective compounds are prepared by the methods of solvent extraction, water-phase organic extraction, crystallization purification, combined with the method of chromatography without employment of the traditional acid-base organic-solvent extraction. FIG. 1 and FIG. 2 show the structural formula of dehydrocavidine and dehydroapocavidine. According to the chemical properties and solubility of dehydrocavidine and dehydroapocavidine, dehydrocavidine-dehydroapocavidine composition and their respective compounds are prepared by the methods of solvent extraction, water-phase organic extraction, crystallization purification, combined with the method of chromatography without employment of the traditional acid-base organic-solvent extraction.

A method proposed in the present invention for preparing a dehydrocavidine-dehydroapocavidine composition, and their respective individual compounds comprises the following steps: isolate and purify the quaternary ammonium components from medicinal material of Yan Hung Lian (Corydalis saxicola Bunting) via solvent extraction, water-phase organic extraction, crystallization and recrystallization, and then dry to obtain the dehydrocavidine and dehydroapocavidine compositions which can be further separated by chromatography to obtain individual compounds of dehydrocavidine and dehydroapocavidine.

Said medicinal material of Yan Huang Lian (Corydalis saxicola Bunting) may be freshly collected raw medicinal material or commercially available medicinal material. The content of dehydrocavidine within the medicinal material should reach a certain level, and it provides that only the medicinal material with dehydrocavidine content above 0.5 percent can be used as the preparation material; if the dehydrocavidine content is too low, the yield rate of the products cannot be guaranteed.

When using said solvent extraction, the solvents can be organic solvents such as water, acidic water, methanol, ethanol, propanol, butanol and ethyl acetate, or their mixture; the extraction methods can be ultrasonic extraction, percolation extraction or reflux extraction; the extraction can be repeated more than one time.

When using said water-phase organic extraction, the extracts of medicinal materials of Yan Huang Lian (Corydalis saxicola Bunting) can be dispersed in water, defatted with petroleum ether and extracted with appropriate organic solvents in order to remove the non quaternary ammonium alkali species. The organic solvents may be chloroform, dichloromethane, ether, ethyl acetate, butanol, etc. for this extraction. The extraction may also proceed directly with above solvents without defatting with petroleum ether. The extraction may be repeated more than one time.

When using the methods of crystallization, the extraction residues obtained from the water-phase extraction preparation are crystallized with an appropriate solvent in order to remove the inorganic salts and fractional non-quatemary ammonium alkali species. The crystallization solvents may be water, methanol, ethanol, butanol, acetone, etc., and the mixed solvent of two or more above solvents. The solvent can be used at a low temperature, room temperature or slightly heated.

When using the methods of recrystallization, the crude quaternary ammonium alkali species of Yan Huang Lian (Corydalis saxicola Bunting) obtained from the crystallization are dissolved by heating with appropriate solvents, and are filtrated and concentrated, then the solution is placed at a low temperature and the dehydrocavidine-dehydroapocavidine composition can be separated out from the solution. The solvents may be used individually or in a mixture of the following: methanol, ethanol, water, acidic water, acidic methanol, acidic ethanol. The acid used may be hydrochloric acid, sulfuric acid, phosphoric acid, nitric acid, perchloric acid, succinic acid, oxalic acid, formic acid, acetic acid or their mixture. The crystallization can be repeated more than once.

The drying method of said dehydrocavidine-dehydroapocavidine composition is atmospheric pressure drying or decompression drying, and may also be spray drying or freeze drying. The contents of dehydrocavidine and dehydroapocavidine are in the range of 5% to 99.5% (w/w) of the composition obtained from said methods.

Chromatography is used to obtain individual compounds of dehydrocavidine and dehydroapocavidine by separating the dehydrocavidine-dehydroapocavidine composition and crude extracts obtained from each step of the composition preparation. The chromatographic packing can be used individually or a combination of silica gel, aluminum oxide, polyamide, sephadex gel. The chromatography can be a column or a thin layer.

Experiments have demonstrated that the dehydrocavidine-dehydroapocavidine composition and their respective individual compounds have anti-hepatitis B virus activity and a protective effect on liver injury, and that they can promote the detoxification function of the liver, and also can protect the liver against chemical poisons. Their potency is superior to that of matrine.

The experiments have also demonstrated that the dehydrocavidine-dehydroapocavidine composition and their respective compounds have an inhibitory effect on the human telomerase, inhibit tumor growth, inhibit virus activity and inhibit arrhythmia.

The pharmaceutical composition for clinical therapy can be manufactured by using the dehydrocavidine-dehydroapocavidine composition and their individual compounds with the addition of one or more pharmaceutically acceptable excipients, which can be used to treat acute and chronic virus hepatitis, liver injury, influenza, tumors, AIDS and arrhythmia.

The pharmaceutically acceptable excipients are the regular excipients routinely used in pharmaceutical industry, for example, diluent, vehiculum such as water; filler material such as starch, sucrose; binder such as cellulose derivatives, alginate, glutin and polyvinylpyrrolidone; lubricators such as glycerin; disintegrants such as agar, calcium carbonate, sodium bicarbonate; absorption accelerators such as quaternary ammonium compounds; surfactants such as cetanol; adsorption matrices such as kaoline, bentonite; malactics such as tarcum powder, calcium and magnesium stearate, polyethylenepolythene. In addition, other supporting agents such as fragrances and sweetening agents can also be added.

The compounds can be administrated to the patients through oral administration, nasal inhalation, rectum or external rectum administration. For oral administration, the compounds can be prepared into solid preparations such as a tablet, powder, granules, capsules, and in liquid preparations such as water or oil-suspending agents or other liquid preparations such as syrups, elixirs; for external rectum administration, the compounds can be prepared into injection solution, water or oleo-suspending agents. The optimum preparations are tablet, coated tablet, capsule, suppository, nasal pressurized spray and injection solution.

All kinds of preparations of the pharmaceutical composition can be prepared by the methods routinely used in the field of pharmacology. For example, the active constituents can be combined with one or more excipients and then prepared to the desired preparations.

Employment of the method disclosed in the present invention can greatly increase the yield rate and transfer rate. The dehydrocavidine-dehydroapocavidine composition and their respective individual compounds prepared in said method have high content of quaternary ammonium alkaloids and a relatively stable ratio of the active constituents. They can be used for manufacturing pharmaceutical medicines to fight hepatitis, viruses, tumors and arrhythmia.

Examples of practices below are provided to help professionals in this field understand the invention, but are by no means intended to limit the scope of the present invention.

### BRIEF DESCRIPTION OF THE DRAWINGS

FIG. 1 is the structural formula of dehydrocavidine;

FIG. 2 is the structural formula of dehydroapocavidine;

FIG. 3 is the HPLC chromatogram of standard dehydrocavidine;

FIG. 4 is the HPLC chromatogram of dehydrocavidine-dehydroapocavidine composition;

FIG. 5 is the HPLC chromatogram of dehydrocavidine compound; and

FIG. 6 is the HPLC chromatogram of the self-prepared dehydroapocavidine compound.

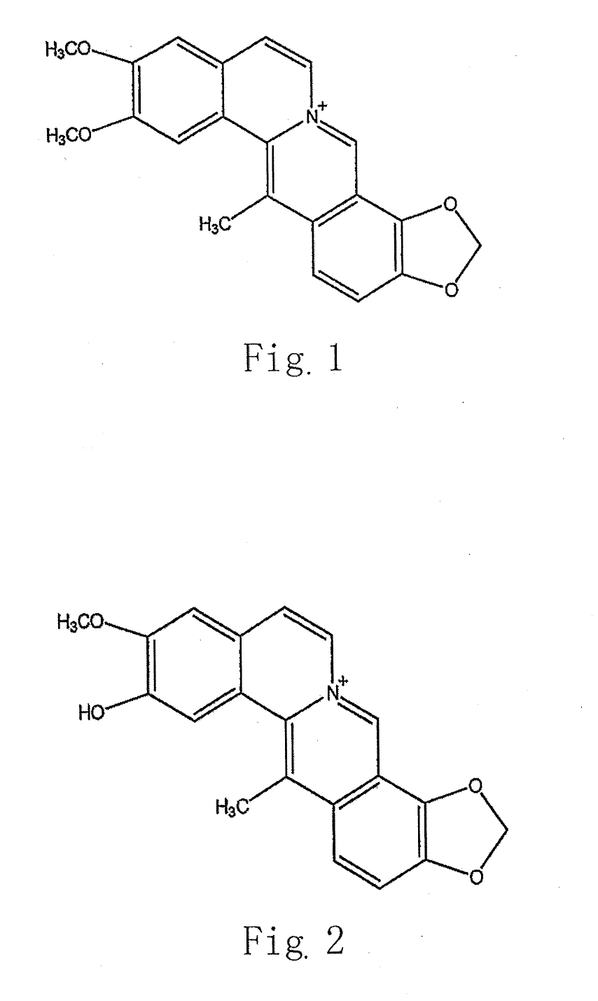

### DETAILED DESCRIPTION OF THE PREFERRED EMBODIMENTS

### Preferred Embodiment 1

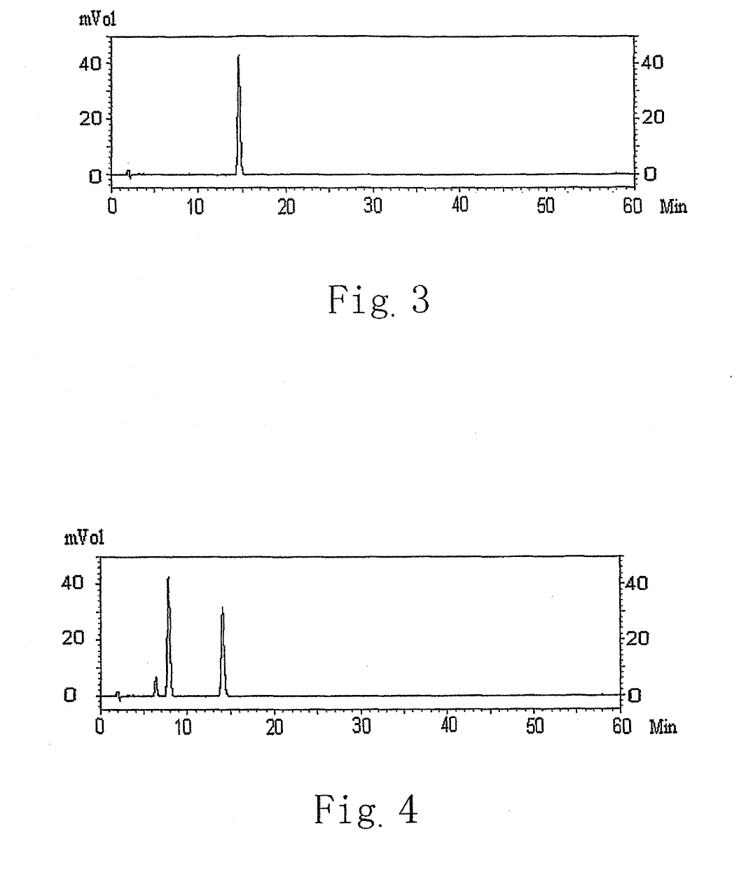

2.5 kg of the dried medicinal plant Yan Huang Lian (Corydalis saxicola Bunting) are reflux extracted twice (2 hour one time) in 50 liters of 90 percent ethanol, and the extractive solution combined is concentrated to 580 g extracts. The extracts are dissolved in 2 liters of water, defatted by three times of extraction in 6 liter petroleum ether followed by three times of extractive purification in 6 liter chloroform. The extractive residues are gauze filtered to remove the solution and then are respectively washed by 2 liters of water and 2 liters of ethanol, and 62 g of crude extract are obtained after being filtered through gauze. The crude extract is dissolved in 1 liter of 1 percent hydrochloric acid ethanol solution, and hot filtration is performed. The filtrate is placed in a refrigerator at 4.degree. C. overnight, and consequently, the dehydrocavidine-dehydroapocavidine composition crystals are separated out. 36 g of purified composition is obtained after being filtered and dried. The chromatogram of HPLC is shown in FIG. 4. Standards of dehydrocavidine are shown in FIG. 3.

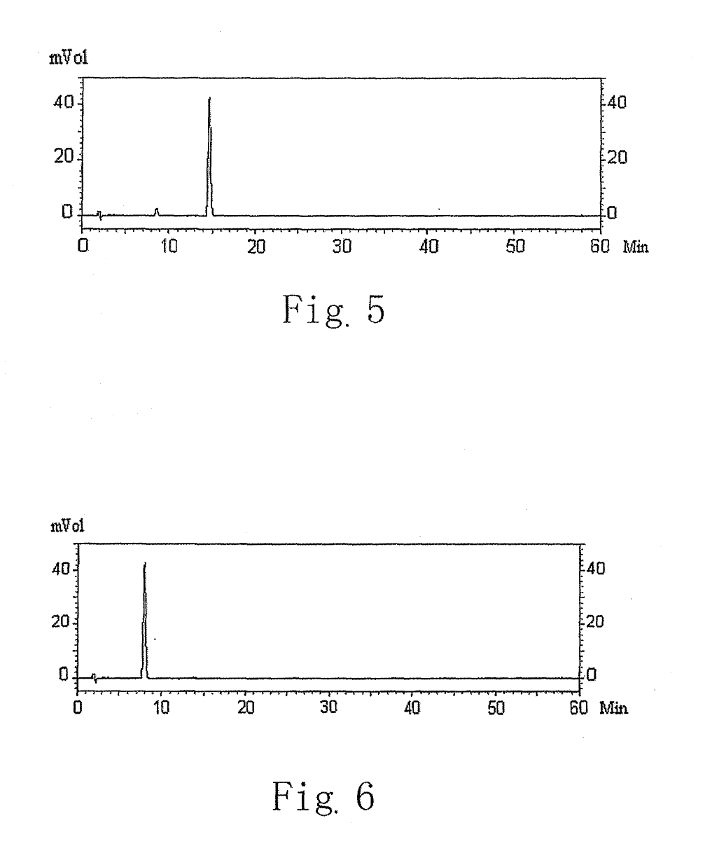

10 g of the dehydrocavidine-dehydroapocavidine composition is mixed with 50 g silica gel, and is then added to the top of a silica column, and a gradient elution is performed with a solvent system of chloroform:methanol (15:1.about.5:1) accompanied by TLC tracking. The eluate containing dehydrocavidine is combined and then concentrated by decompression to dry, resulting in a 3.9 g dehydrocavidine compound and a 3.7 g dehydroapocavidine compound respectively. The chromatograms of HPLC are showed in FIGS. 5 and 6.

### Preferred Embodiment 2

Measuring the content of dehydrocavidine in the medicinal plant Yan Huang Lian (Corydalis saxicola bunting)

Measuring the content of dehydrocavidine in the medicinal plant Yan Huang Lian (Corydalis saxicola Bunting) is performed by high performance liquid chromatography as shown in the Pharmacopoeia of China, and the methods and its chromatographic conditions are as follows:

Chromatographic conditions and the system applicability test: the packing material is octadecyl silane bonded silica gel (pls double check this term translated) and the mobile phase is A. Phosphoric acid saline buffer solution (each liter contains 20 mmol KH2PO4, 10 mmol triethylamine and 0.2% of phosphoric acid; B. ethyl nitrile. Employing A:B (78:22) as the mobile phase, the column temperature is at 30.degree. C., the wavelength is 347 nm, and the flow rate is 0.8 ml/min and the sample collection time is 30 min. The value of the height equivalent to a theoretical plate related to dehydrocavidine should not be lower than 3000.

Preparation of the solvent for a standard sample: weigh precisely 10 mg of the standard sample of dehydrocavidine and place in a 100 ml measuring beaker; then add the mobile phase A:B (78:22) into the beaker up to the planned scale and shake well and save it as stock solution.

Measure precisely 5.0 ml of said stock solution and place it in a 25 ml measuring beaker, and then add the mobile phase (A:B=78:22) up to the planned scale and shake well. It can then be prepared as a solvent of 20.mu.g in 1 ml.

Preparation of solvent for test sample: grind the medicinal plant Yan Huang Lian (Corydalis saxicola Bunting) and sift it out with #20 sieve (0.850 mm screen aperture). Obtain 100 mg powder and weigh precisely, then place it in a 50 ml measuring beaker. The mobile phase (A:B=78:22) is added into the beaker up to the planned scale and is then ultrasonically treated for 30 minutes. It is then filtered out with micropore filtering membrane. The filtrate is obtained as the test sample.

Method for measurement: precisely 10.mu.l standard sample and test sample are pipetted out respectively and are loaded onto liquid chromatography. The content can be measured with the external standard method by calculating the peak area.

The results of the measurement of fresh and commercially available medicinal plant Yan Huang Lian (Corydalis saxicola Bunting) in accordance with said standards are shown in the following table:

| Code | Sources | Dehydrocavidine (%) |
|---|---|---|
| 1 | Collected at Hechi, Guangxi - 1 | 0.80 |
| 2 | Collected at Hechi, Guangxi - 2 | 0.72 |
| 3 | Collected at Hechi, Guangxi - 3 | 0.76 |
| 4 | Purchased at the medicinal market | 0.68 |
| | of Haozhou, Anhui | |
| 5 | Purchased at the medicinal market | 0.72 |
| | of Anguo, Hebei | |

According to the results of the above measurements, the limit for dehydrocavidine content should not be set below 0.5 percent. Only medicinal materials that meet this standard can be used.

### Preferred Embodiment 3

2.5 kg fresh medicinal plant of Yan Huang Lian (Corydalis saxicola Bunting) are ultrasonically extracted twice for 1 hour at a time, and the extractive solution combined is concentrated by decompression to 415 g extracts. The extracts are dissolved in 2 liters of water, and purified by extracting it three times in 6 liters of dichloromethane. The extractive residues are gauze filtered to remove the solution and are then washed with 2 liters of ethanol, and 53 g of crude extract are obtained after being filtered through gauze. The crude extract obtained is dissolved in 1 liter of 2.5 percent hot acetic acid solution, and hot filtration is performed. The filtrate is placed in a refrigerator to cool overnight, and the dehydrocavidine-dehydroapocavidine composition is then separated out. 27 g of purified composition is obtained after being paper filtered and freeze dried. The content of the dehydrocavidine-dehydroapocavidine composition is measured and the result shows 17 percent of dehydrocavidine and dehydroapocavidine in the composition.

10 g of the dehydrocavidine-dehydroapocavidine composition obtained from the above step is mixed with 40 g of the aluminum oxide, and are then added to the top of an aluminum oxide column, and a gradient elution is employed with a solvent system of petroleum ether: ethyl acetate (1:8.about.1:15) accompanied by TLC tracking. The eluate containing dehydrocavidine is combined and then is concentrated by decompression to constant weight, resulting in a 1.3 g dehydrocavidine compound and a 1.9 g dehydroapocavidine compound respectively.

### Preferred Embodiment 4

2.5 kg of commercially available medicinal plant of Yan Huang Lian (Corydalis saxicola Bunting) are percolationally extracted in 50 liter ethyl acetate, and the percolative solution collected is concentrated by decompression to 475 g extracts. The extracts are dissolved in 2 liters of water, and defatted three times through extraction in 6 liters of petroleum ether, and are then purified three times through extraction in 6 liters of ethyl acetate. The extractive residues are gauze filtered to remove the solution and are then washed respectively by 2 liters of ethanol and 2 liters of acetone. 63 g of crude extract are obtained after being filtered through gauze. The crude extracts obtained are dissolved in 1 liter of 1 percent hot sulfuric acid methanol solution, and hot filtration is performed. The filtrate is placed in a refrigerator to cool overnight, and the dehydrocavidine-dehydroapocavidine composition is subsequently separated out. 29 g of purified composition is obtained after being paper filtered and decompression dried to a constant weight.

The content of the dehydrocavidine-dehydroapocavidine composition is measured and the result shows 99.5 percent of dehydrocavidine and dehydroapocavidine in the composition.

5 g of the dehydrocavidine-dehydroapocavidine composition obtained from the above step is mixed with 20 g polyamide, and are then added to the top of a polyamide column, and a gradient elution is employed with a solvent system of methanol: water (4:1) accompanied by TLC tracking. The eluate containing dehydrocavidine is combined and is then concentrated by decompression to a constant weight, resulting in obtaining 0.9 g of a dehydrocavidine compound and 0.75 g of a dehydroapocavidine compound respectively.

### Preferred Embodiment 5

2.5 kg fresh medicinal plant of Yan Huang Lian (Corydalis saxicola Bunting) are reflux extracted twice for 2 hours at a time in 25 liters of hot water, and the extractive solution is combined and is then concentrated by decompression to 2.5 liter extracts. The extracts are purified four times through extraction in 10 liters of butanol. The extractive residues are gauze filtered to remove the solution and are then washed with 2 liters of water. 31 g of crude extract are obtained after being filtered through gauze. The crude extract obtained is dissolved by heating in 0.5 liters of ethanol and hot filtration is performed. The filtrate is placed in a refrigerator to cool overnight, and the dehydrocavidine-dehydroapocavidine composition is subsequently separated out. 15 g of purified composition is obtained after being filtered with filter paper and decompression dried to a constant weight.

The content of the dehydrocavidine and dehydroapocavidine in the dehydrocavidine-dehydroapocavidine composition is measured and the result shows 5.01 percent of dehydrocavidine and dehydroapocavidine in the composition.

200 mg of the dehydrocavidine-dehydroapocavidine composition obtained from the above step is dissolved with 10 ml of methanol, and are then loaded to the top of the sephadex gel column, and a gradient elution is employed with 30%.about.70% methanol, accompanied by TLC tracking. The eluate containing dehydrocavidine is combined and is then concentrated by decompression to a constant weight, resulting in obtaining 22 mg of a dehydrocavidine compound and 14 mg of a dehydroapocavidine compound respectively.

### Preferred Embodiment 6

0.5 kg of commercially available dried medicinal plant of Yan Huang Lian (Corydalis saxicola Bunting) are refluxly extracted twice for 1 hour at a time in 5 liters of propanol, and the extractive solution is combined and is then concentrated by decompression to 105 g extracts. The extracts are mixed with 150 g of silica gel and are loaded to the top of the silica gel column, and a gradient elution is employed with a solvent system of chloroform: methanol, accompanied by TLC tracking. The eluate with the same spot containing dehydrocavidine is combined and is then concentrated by decompression to a constant weight, resulting in obtaining 1.9 g dehydrocavidine compound and a 1.4 g dehydroapocavidine compound respectively.

### Preferred Embodiment 7

0.5 kg of fresh medicinal plant of Yan Huang Lian (Corydalis saxicola Bunting) are extracted through percolation in 10 liters of a 1 percent hydrochloric acid solution, and the extractive solution is collected and is then concentrated by decompression to 95 g extracts. The extracts are dissolved in 1 liter of water, and are then purified three times through extraction in 3 liters of dichloromethane. The extractive residues are gauze filtered to remove the solution. 47 g of extracts are obtained after being filtered through gauze and dried by decompression. The extracts are mixed with 100 g of polyamide and are loaded to the top of the polyamide column, and a gradient elution is employed with a solvent system of ethanol:water (1:10.about.1:1), accompanied by TLC tracking. The eluate of the same band containing dehydrocavidine is combined and is then concentrated to a constant weight, resulting in obtaining 2.3 g of a dehydrocavidine compound and 1.8 g of a dehydroapocavidine compound respectively.

### Preferred Embodiment 8

1 kg of fresh medicinal plant Yan Huang Lian (Corydalis saxicola Bunting) are ultrasonically extracted twice for two hours at a time in 20 liters of butanol, and the extractive solution is combined and is then concentrated by decompression to 156 g extracts. The extracts are dissolved in 1.5 liters of water, and defatted by extraction four times in 6 liters of petroleum ether, and are then purified by extraction four times in 6 liters of ethyl acetate. The extractive residues are gauze filtered to remove the solution and are then washed respectively in 2 liters of ethanol and 2 liters of acetone. 63 g of extracts are obtained after being filtered through gauze and dried by decompression. 200 mg of the extract is dissolved in 10 ml of methanol and is then loaded to the top of the sephadex gel column. A gradient elution is used with methanol, accompanied by TLC tracking. The eluate in the same band containing dehydrocavidine is combined and is then concentrated by decompression to a constant weight, resulting in obtaining 23 mg of a dehydrocavidine compound and 15 mg of a dehydroapocavidine compound respectively.

### Preferred Embodiment 9

1 kg of commercially available dried medicinal plant Yan Huang Lian (Corydalis saxicola Bunting) are extracted by reflux two times, each for one hour at a time in 10 liters of 70 percent methanol, and the extractive solution is collected and is then concentrated by decompression to 1 liter of extracts. The extracts are purified by extracting them three times in 3 liters of chloroform. The extractive residues are gauze filtered to remove the solution and are then washed with 1 liter of ethanol. 23 g of crude extract are obtained after being filtered through gauze. 50 mg of the crude extracts is dissolved in 2 ml methanol and 0.5 ml sample from the dissolved extracts is loaded to the sheet with a thin layer of silica gel. A developing solvent of ethyl acetate:methanol:isopropanol:ammonia (30:15:15:7.5:1.5) is employed and the sample is developed in the chromatographic chamber. The gel with corresponding bands to dehydrocavidine and dehydroapocavidine is peeled off from the sheet and transferred into a flask, which is then ultrasonically extracted in an appropriate amount of methanol. The extracts obtained are filtered and concentrated by decompression to dry. 11 mg and 7 mg of individual compounds of dehydrocavidine and dehydroapocavidine are obtained respectively.

### Preferred Embodiment 10

| | | |
|---|---|---|
| Tablet: | Active constituents | 10 mg |
| | Lactose | 187 mg |
| | Cornstarch | 50 mg |
| | Magnesium stearate | 3 mg |

Preparation: the mixture of active constituents, lactose and cornstarch is wetted with water. The wetted mixture is then sifted out and dried. The dried mixture is sifted out again. Then the mixture is compressed to tablets (250 mg/tablet) after adding magnesium stearate. The content of active constituents is 10 mg per tablet.

### Preferred Embodiment 11

| | | |
|---|---|---|
| Intravenous drip: | Active constituents | 2 mg |
| | Sodium chloride | 9 mg |

Preparation: the active constituents and sodium chloride are dissolved in appropriate amount of fluid for injection. The solution is filled in a container in aseptic condition after being filtered.

### Preferred Embodiment 12

The inhibitory effects of dehydrocavidine-dehydroapocavidine composition and their respective compounds on hepatitis viruses

The inhibitory effects of said dehydrocavidine-dehydroapocavidine composition and their respective compounds on hepatitis B viruses are determined using the cell line 2.2.15 of human liver cancer cell (Hep G2) transfected by hepatitis B virus DNA. The results are showed in Table 1.

TABLE 1
The toxicity of dehydrocavidine-dehydroapocavidine composition
and their respective compounds to cell Hep G2 2.2.15 and their
inhibitory effects on HBeAg, HBsAg

| | | | Inhibition | Inhibition |
|---|---|---|---|---|
| | | Toxicity | frequency | frequency |
| | Concentration | to | to HBeAg | to HBsAg |
| Samples | (μmol/ml) | cell | (%) | (%) |
| Dehydrocavidine | 0.4 | − | 57.2 | 34.6 |
| | 0.2 | − | 21.6 | 31.4 |
| | 0.1 | − | 20.5 | 28.4 |
| dehydroapocavidine | 0.4 | − | 62.1 | 28.6 |
| | 0.2 | − | 33.5 | 21.3 |
| | 0.1 | − | 26.5 | 15.7 |
| composition | 0.4 | + | / | / |
| | 0.2 | − | 50.1 | 32.3 |
| | 0.1 | − | 44.1 | 19.6 |
| 3TC | 1.0 | − | 24.5 | 4.8 |

−: “no obvious toxicity” means cell livability ≧75% using MTT method;
+: “showing toxicity” means cell livability ≦75%.

### Preferred Embodiment 13

The animal verification on protective effects of dehydrocavidine-dehydroapocavidine composition and their respective compounds on experimental liver injury

The model of acute liver injury induced by thioacetamide in mice

Experimental animals: Kunming mice, male or female, weighing 19-22 g, are used in the study. Animals are housed in an animal room with a natural photoperiod and room temperature (23.+−0.2.degree. C.), and maintained with free access to standard rodent pellet food and water ad libitum.

1.2 Experimental methods: All mice are randomly divided to 7 groups. All the other groups are treated with 30 mg/kg thioacetamide to induce acute liver injury model except the normal control group. Then all groups are successively dosed three times at an interval of 3 hours, 6 hours and 9 hours after being infected, and the blood samples were obtained at 24 hours (i.e. the next day) after the final dose. The follow markers and liver weight were measured and also the pathological examination were performed (Table 2).

TABLE 2
The effects of tested reagents on the markers of the
model of acute liver injury induced by thioacetamide
in mice (X ± SD, n = 20)

| | | | Liver index |
|---|---|---|---|
| | | | (g liver wt./100 g |
| Groups | ALT | AST | body wt.) |
| Dehydrocavidine | 873.50 ± 251.82^{††} | 237.10 ± 73.27^{††} | 5.36 ± 0.54 |
| Dehydroapocavidine | 823.26 ± 178.42^{††} | 241.13 ± 62.81^{††} | 5.14 ± 0.48 |
| Composition | 924.72 ± 173.23 | 271.98 ± 123.82 | 4.93 ± 0.44 |
| Positive contrast | 885.30 ± 248.27^{†} | 293.30 ± 175.27^{†} | 4.69 ± 0.58 |
| NS saline | 1183.70 ± 238.53 | 366.10 ± 71.42 | 4.65 ± 0.51 |
| Control | 701.30 ± 117.25 | 293.60 ± 62.75 | 4.78 ± 0.50 |

*compared with control group:
^{†}P < 0.05;
^{††}P < 0.01

Results: In contrast with the control group, the dehydrocavidine species chemicals can markedly relieve acute liver injury induced by thioacetamide in mice.

2. The Model of Acute Hepatic Toxicity Induced by Carbon Tetrachloride in Rats

2.1 Experimental Animals

Matured SD rats, male or female, weighing 250-350 g are used in the study. Animals are maintained with free access to standard rodent pellet food and water ad libitum.

2.2 Experimental Methods

All rats are randomly divided into 6 groups. All the other groups are treated with 0.5 ml/100 g of 50 percent carbon tetrachloride by hypodermic injection to induce acute hepatic toxicity model, except the control group, and are dosed one time simultaneously. Then all groups will be successively dosed at 4 hours and 8 hours, and the blood samples obtained at 12 hours after the final dose for determining ALT and AST. The animals are killed to measure their liver weight and to perform a pathological examination (Table 3).

TABLE 3
The effects of tested reagents on certain markers of the
model of acute hepatic toxicity induced by carbon
tetrachloride in rats (X ± SD, n = 20)

| Groups | ALT | AST |
|---|---|---|
| Dehydrocavidine | 774.30 ± 217.11^{††} | 1116.54 ± 348.27^{††} |
| Dehydroapocavidine | 955.36 ± 327.18^{††} | 903.52 ± 256.94^{††} |
| Composition | 1083.22 ± 824.61^{††} | 1083.12 ± 526.81^{††} |
| NS saline | 4865.18 ± 212.33 | 4126.54 ± 245.37 |
| Positive contrast | 1435.27 ± 235.62 | 1382.23 ± 173.36 |
| Control | 926.73 ± 121.52 | 913.83 ± 139.77 |

*compared with negative control group:
^{†}P < 0.05;
^{††}P < 0.01

Results: In contrast with the negative control group, the dehydrocavidine species chemicals can markedly relieve the acute hepatic toxicity induced by carbon tetrachloride in rats.

3. The Model of Hepatic Toxicity Induced by D-Galactosamine in Rats

3.1 Experimental Animals

Matured SD rats, male or female, weighing 250-350 g are used in study. Animals are maintained with free access to standard rodent pellet food and water ad libitum.

3.2 Experimental Methods

All rats are randomly divided into 4 groups. All groups except the control group are treated simultaneously with 800 mg/kg of 50 percent D-galactosamine by intraperitoneal injection to induce acute hepatic toxicity model. The blood samples are obtained 12 hours after the final dose for determining SGPT and TBIL. The animals are killed to measure their liver weight and to perform a pathological examination (Table 4).

TABLE 4
The effects of tested reagents on the model of acute
hepatic toxicity induced by carbon tetrachloride in rats
(X ± SD, n = 20)

| | | | Liver index |
|---|---|---|---|
| | | | (g liver wt./100 g |
| Groups | ALT | AST | body wt.) |
| Dehydrocavidine | 846.28 ± 172.54^{††} | 241.20 ± 65.53^{††} | 5.10 ± 0.36 |
| Dehydroapocavidine | 1027.10 ± 277.38 | 345.20 ± 68.29 | 4.47 ± 0.47 |
| Composition | 829.17 ± 103.42 | 338.21 ± 58.82 | 4.01 ± 0.25 |
| Positive contrast | 837.28 ± 172.63^{††} | 284.39 ± 171.92 | 4.72 ± 0.44 |
| NS saline | 1204.30 ± 219.78 | 383.27 ± 67.28 | 4.79 ± 0.51 |
| Control | 726.54 ± 103.36 | 228.18 ± 37.22 | 4.63 ± 0.32 |

*compared with control group:
^{†}P < 0.05;
^{††}P < 0.01

4. The Model of Liver Fibrosis Induced by Carbon Tetrachloride in Rats

4.1 Experimental Animals

Wistar male rats, weighing 100-150 g are used in the study. Animals are housed in an animal room with a 12 h:12 h photoperiod and at 22.degree. C. Animals are maintained with free access to standard rodent pellet food and water ad libitum

4.2 Experimental Methods

All Wistar male rats are treated with 0.3 ml/(100 g body weight) of 40 percent carbon tetrachloride (dissolved in peanut oil) by hypodermic injection twice a week for 12 weeks to induce acute hepatic fibrosis model, all in phase IV. Then all rats in hepatic fibrosis are randomly divided into the following groups: normal saline (NS) group, sample group and positive contrast group. The NS group rats are treated with 0.2 ml normal saline by intramuscular injection for 8 weeks, and simultaneously, all the other groups are treated with samples one time a day for 8 weeks. The blood samples are obtained from the inferior vena cava 8 weeks later for determining biochemical markers. After that, all rats are killed, and the right lobes of the livers are fixed in neutral formaldehyde solution for histological examination. The results are shown in Table 5.

TABLE 5
The effects of tested reagents on the model of chronic
hepatic toxicity induced by carbon tetrachloride in rats
(X ± SD, n = 20)

| Groups | ALT | AST | Hydroxyprodine in Liver |
|---|---|---|---|
| Dehydrocavidine | 1113.4 ± 247.6 | 987.3 ± 237.9 | 0.162 ± 0.013 |
| Dehydroapocavidine | 973.2 ± 273.5 | 1057.1 ± 338.7 | 0.175 ± 0.018 |
| Composition | 1113.4 ± 247.6 | 987.3 ± 237.9 | 0.162 ± 0.013 |
| NS saline | 2284.2 ± 273.6 | 2949.9 ± 1572.4 | 0.195 ± 0.024 |
| Positive contrast | 1644.7 ± 158.3 | 1834.6 ± 836.4 | 0.217 ± 0.040 |
| Control | 989.6 ± 180.8 | 1085.3 ± 437.7 | 0.169 ± 0.018 |

*compared with negative control group:
† P < 0.05;
†† P < 0.01

Results: In contrast with the control group, the dehydrocavidine species chemicals can markedly relieve the chronic hepatic toxicity induced by carbon tetrachloride in rats.

### Preferred Embodiment 14

The inhibitory effects of dehydrocavidine-dehydroapocavidine composition and their respective compounds on telomerase activity

The lead compounds with the inhibitory effect on telomerase activity are preliminary screened from the potent components of Chinese herbal medicine by a cell-free system. The telomerase proteins are extracted from tumor cells whose telomerase activity showed positive. The plant effective constituents' effects on telomerase activity are tested by the standard method of Telomeric Repeat Amplification Protocol (TRAP) which is the standard method for testing telomerase activity. All effective constituents (10-100.mu.mol) are incubated with the extracts from tumor cells for a specific amount of time (10-20 min), then TRAP testing is performed and the IC50 are calculated. The results are shown in Table 6.

TABLE 6
The inhibitory effects of dehydrocavidine species
compounds on telomerase

| Groups | IC₅₀ (mmol) |
|---|---|
| dehydrocavidine | 17 |
| dehydroapocavidine | 10 |
| composition | 19 |

—: “no inhibition activity” means IC₅₀ ≧100 mmol by TRAP method.

### Preferred Embodiment 15

The inhibitory effects of dehydrocavidine-dehydroapocavidine composition and their respective compounds on HIV viruses

Tested Reagents

Preparation of samples and solvents: the tested reagents are prepared in DMSO according to the planned concentration. Conservation: 4.degree. C. AZT (zidouvding) serves as a positive control sample.

1.2 Cell and Virus

HIV-1 III B comes from the USA; MT4 cell line comes from Japan.

1.3 The Toxicity Experiments of Compounds on Cells

MT4 cells are cultured in 96-well plates (2.times.105 cells/ml, 0.1 ml/well) and the verifying compounds are added and compared against the positive control sample AZT, and the normal cell control group at the same time. Comparisons against DMSO control group and MT4 cell control group are also performed. The culture is maintained at 37.degree. C., 5 percent CO2 for 6 days. The cell activities are tested by the MTT method to determine IC50.

1.4 The Inhibitory Effects of the Compounds on HIV-Induced MT4 Cell Pathological Effects

To determine viral toxicity, HIV are diluted 10 fold in 8 serial [Chinese version is unclear, and this is an educated guess] and MT4 cell pathological effects are then observed in the culture solution of RPMI-1640. Calculated TCID50 is 10-6. The normal cell control group and virus control group are also performed. The samples with five concentrations per group that are diluted by 2 times and 100.mu.l AZT are added in cell or virus cultures. All concentrations of samples are performed in three repeated wells. The experiments are maintained at 37.degree. C., 5 percent CO2 for 72 hours. Then the cell pathological effects (CPE) are observed under an inverted microscope. IC50 and selective index SI (TC50/IC50) are calculated in Table 7.

TABLE 7
The inhibitory effects of dehydrocavidine
species compounds on HIV viruses

| Groups | IC₅₀ (μg/ml) | TC₅₀ (μg/ml) | SI |
|---|---|---|---|
| dehydrocavidine | 12.5 | >1000 | >80 |
| dehydroapocavidine | 6.25 | 250 | 40 |
| composition | 12.5 | 500 | 40 |
| AZT | 0.1 | 500 | 5000 |

Note:
IC₅₀ is 50 percent effective concentration;
TC₅₀ is 50 percent non-toxic concentration;
SI is the selective index;
— means no effect.

Results: The dehydrocavidine, dehydroapocavidine and their respective composition all have certain inhibitory effects on HIV viruses

### Preferred Embodiment 16

The inhibitory effects of dehydrocavidine-dehydroapocavidine composition and their respective compounds on influenza viruses

### Tested Reagents

Preparation of samples and solvents: the tested reagents are prepared in DMEM medium according to the planned concentration. Conservation: 4.degree. C. Ribavirin served as the positive control reagent.

1.2 Cell and Virus

MDCK (Madin darby canin kidney) cell and influenza A1 virus are purchased from the Institute of Virology, Chinese Academy Preventive Medicine (Beijing, China).

1.3 Preparation of MDCK Cell Growth Medium, Cell Maintenance Medium, Versense Solution and Digestive Juice.

Prepared as shown in the cited literature (Guo Yuanji, Cheng Xiaowen, 1997).

2 Experimental Methods

2.1 MDCK Cells Subculture and Influenza Viruses Culture: Performed in Accordance with the Methods Shown in the Cited Literature by Guo Yuanji, Cheng Xiaowen, 1997.

2.2 Cell Toxicity

The sample (0.1 ml/well) is added on the cell plates that are covered with a layer of cells, subsequently, the cell maintenance medium is added in until the final volume 1 ml/well. The cells are maintained at 37.degree. C., 5 percent CO2 for 72 hours. The cell pathological effects (CPE) are observed under an inverted microscope in contrast with the MDCK cell. All experiments are repeated 2 times. The results indicate that samples do not have nonspecific cell pathological effects (CPE) on MDCK cells.

2.3 Anti-Influenza Virus Experiment

MDCK cells are cultured in 96-well cell culture plates. A normal cell control group, a virus control group, a positive control group and a testing group are set up in the experiment. The influenza A1 viruses are added in the virus control group and the testing group at 37.degree. C. for 2 hour absorption, and are then removed. The samples with different concentrations are respectively added in each group. The experiments are maintained at 37.degree. C., 5 percent CO2 for 3 days. The result of the experiment is then observed and the different compounds of the 50 percent inhibitory concentration of IC50 on viruses are displayed in Table 8.

TABLE 8
The inhibitory effects of dehydrocavidine species
compounds on influenza viruses

| Groups | IC₅₀ (mmol/L) |
|---|---|
| dehydrocavidine | 7.4 |
| dehydroapocavidine | 4.2 |
| Composition | 5.1 |
| Ribavirin | 3.2 |

Results: The dehydrocavidine, dehydroapocavidine and their composition all have markedly inhibitory effects on influenza viruses.

### Preferred Embodiment 17

The antagonistic effects of dehydrocavidine-dehydroapocavidine composition and their respective compounds on arrhythmia

The antagonistic effects of the dehydrocavidine species compounds on arrhythmia induced by aconitine

Tested Samples

The samples are dissolved in hot normal saline up to the needed concentration. Normal saline serves as the control solution. Propafenone serves as a positive control sample.

1.2 Experimental Methods:

Wistar rats of both sexes are randomly grouped and anesthetized by urethane (1.2 g/kg), II lead electrocardiogram is recorded. The groups are administered the following drugs by vena femoralis injection: (1) the compound (5 mg/kg, if possible 2.5 mg/kg), (2) propafenone (7 mg/kg), (3) control solution (2 mg/kg). After 5 minutes, aconitine solution (5.mu.g/ml) was delivered intravenously at a constant speed of 0.08 ml/min. The volume of aconitine solution is recorded when VP, VT and VF occur, and the electrocardiogram is also recorded. EV50 (VF) values are calculated by regression analysis on the basis of the dosage of aconitine consumed by VF in each experiment. The results are shown in Table 9.

TABLE 9
The ED₅₀ of the dehydrocavidine species compounds

| Groups | ED₅₀ (10⁻⁶ mol/Kg) |
|---|---|
| dehydrocavidine | 7.33 |
| dehydroapocavidine | 4.26 |
| Composition | 5.17 |

The phenomenon that the dosage of aconitine is increased and the emerging time of VT and (or) VF is postponed after the tested compounds are intravenously injected, indicate that dehydrocavidine, dehydroapocavidine and the dehydrocavidine-dehydroapocavidine composition all have certain preventive effects on arrhythmia induced by aconitine. The phenomenon that the dosage of aconitine is increased and the emerging time of VT and (or) VF is postponed after the tested compounds are intravenously injected, indicate that dehydrocavidine, dehydroapocavidine and the dehydrocavidine-dehydroapocavidine composition all have certain preventive effects on arrhythmia induced by aconitine.

1. Dehydrocavidine Protection Effect on the D-Glactosamine Hydrochloride-Induced Acute Liver Injury of Rats

Firstly, inject D-glactosamine hydrochloride into the abdominal cavities of matured SD rats by a dose of 500 mg/kg to induce acute liver injury. Beforehand, inject dehydrocavidine into the abdominal cavities of the rats respectively by doses of 0.3, 1.0 and 3.0 mg/kg/d for a week. The ALT activity of the group injected with a lower dose of dehydrocavidine is lower than that of the NS control group by 26.6%. However, the different is not obviously significant. The ALT activities of the groups injected with medium and high doses of dehydrocavidine are respectively lower than that of the NS control group by 44.2% and 37.5%, and P<0.05 for both. The AST activities of the groups injected with low and medium doses of dehydrocavidine are respectively lower than the AST activity of the NS group—5667.1±3122.1 nmol/s/L by 19.1% and 7.1%. However, the differences are not obviously significant. The histopathological examination of the liver tissues shows that providing dehydrocavidine beforehand can reduces extend of acidophilic denaturation, adipose denaturation, dropsy, and multiple-fragmental necrosis of liver cells. The results of the experiment show that the low-dose and medium-dose abdominal-cavity dehydrocavidine injections can decrease extend of ALT and AST rising resulting from the D-glactosamine hydrochloride-induced acute liver injury and thus reduce the pathological change. Therefore, dehydrocavidine can considerably protect rats from the D-glactosamine hydrochloride-induced acute liver injury.

2. Dehydrocavidine protection effect on the carbon tetrachloride-induced chronic liver injury of rats

Inject 25% carbon tetrachloride olive oil solution into rats subcutaneously by a dose of 2 ml/kg two times a week for three months. In the last two months, inject dehydrocavidine into the abdominal cavities of the rats respectively by doses of 0.35, 0.70, 1.40 mg/kg each day for eight weeks to observe the protection effect of dehydrocavidine on the carbon tetrachloride-induced chronic liver injury. The experimental results show that the ALT activities of the groups injected with medium and high doses of dehydrocavidine are lower than the ALT activity of the NS control group—5645±2452 nmol/s/L respectively by 28.4% and 49.2% with P<0.05 and P<0.01 separately. However, the difference between the low-dose dehydrocavidine group and the NS control group is not obviously significant. The GOT activities of the poisoned groups are over two times that of the normal control group. In comparison with the NS group, dehydrocavidine does not obviously influence the activity of GOT. Hydroxyproline concentrations of the groups of the low, medium, and high doses of dehydrocavidine are lower than that of the NS control group—0.22±0.04 μg/mg respectively by 21.2%, 18.9% and 17.5% with P<0.01 for all of them. The histopathological evaluation counts of the groups of the low, medium, and high doses of dehydrocavidine are lower than that of the NS group—4.0±0 respectively by 40.6%, 43.8% and 53.9% with P<0.01 for all of them. Dehydrocavidine does not obviously influence the concentrations of the whole serum protein and albumin. The experimental results show that dehydrocavidine can reduce ALT activity and hydroxyproline concentration and inhibit liver fibrosis. Therefore, dehydrocavidine can prevent from chronic liver injury in a certain extent.

3. Dehydrocavidine Protection Effect on the Phenylisothiocyanate-Induced Liver Injury of Rats

Inject phenylisothiocyanate into the stomachs of male matured SD rats by a dose of 110 mg/kg to induce obstructive jaundice. Beforehand, inject dehydrocavidine into the abdominal cavities of the rats respectively by doses of 0.3, 1.0 and 3.0 mg/kg/d for a week. The ALT activities of the three groups are lower than the ALT activity of the NS control group—2264.0±397.7 nmol/s/L respectively by 27.6%, 31.8% and 44.9% with P<0.01 for all of them. The AST activities of the three groups are lower than the AST activity of the NS control group—6835.0±1357.7 nmol/s/L respectively by 33.7%, 35.3% and 41.6% with P<0.01 for all of them. In comparison with the normal control group, the total bilirubin concentrations of the three dehydrocavidine-injection groups do not rise obviously, but total bilirubin concentration of the NS control group is 122.1% higher than that of the normal control group. Among the three dehydrocavidine-injection groups, the concentrations of the serum conjugated bilirubin of the low-dose and high-dose groups are obviously higher than that of the normal control group with P<0.05 and P<0.01 respectively. The difference between the concentrations of the serum conjugated bilirubin of the medium-dose group and the normal control group is not obviously significant. The concentration of the serum conjugated bilirubin of the NS control group (6.75±2.20 mol/L) is 603% higher than that of the normal control group (0.96±0.46 mol/L) with P<0.01. The concentrations of the serum conjugated bilirubin of the low-dose, medium-dose and high-dose groups are respectively 67.6%, 85.0% and 70.8% lower than that of the NS control group with P<0.01 for all of them. The histopathological examination shows that feeding dehydrocavidine beforehand can decrease the injury of the bile duct epithelial cells and the proliferation of bile ductules. Therefore, dehydrocavidine can relieve the phenylisothiocyanate-induced obstructive jaundice of rats.

4. Influence of Dehydrocavidine on the Bile Secretion of Sd Rats

Inject dehydrocavidine intravenously into SD rats respectively by doses of 0.35, 1.10 and 3.50 mg/kg/d. Observe the influence of dehydrocavidine on the bile secretion of normal SD rats with a bile drainage method. The experimental result shows that all of the low dose, medium dose and high dose of dehydrocavidine can obviously increase the bile secretion of the SD rats. Within one hour of dehydrocavidine injection, the total bile secretions of the three dehydrocavidine-injection groups are respectively 37.4%, 49.5% and 63.7% greater than that of the NS control group—0.91±0.05 ml with P<0.01 for all of them. Within two hour of dehydrocavidine injection, the total bile secretions of the three dehydrocavidine-injection groups are respectively 36.6%, 47.7% and 63.4% greater than that of the NS control group—1.72±0.08 ml with P<0.01 for all of them. The experimental result shows that the intravenous injection of dehydrocavidine can increase the bile secretion of normal SD rats. Therefore, dehydrocavidine can promote the function of gall in a certain extend.

5. Influence of Dehydrocavidine on the Immunological Function of Mouse Body Fluid

The HC50 values of mouse serum hemolysin are used to observe the influence of dehydrocavidine on the immunological function of mouse body fluid. Inject dehydrocavidine into the abdominal cavities of BABL/C mice by doses of 0.5, 1.5 and 5.0 mg/kg/d for four days. The HC50 values of mouse serum hemolysin of the low-dose, medium-dose and high-dose dehydrocavidine-injection groups are respectively 20.8%, 22.1% and 29.8% higher than that of the NS control group—54.4±11.4 with P<0.05 and P<0.01 separately. The experiment result shows that dehydrocavidine favors the generation of SRBC antibody in mice and promotes the immunological function of mouse body fluid.

6. Dehydroapocavidine Protection Effect on the D-Glactosamine Hydrochloride-Induced Acute Liver Injury of Rats

Abstract

Objective: observing the dehydroapocavidine protection effect on the D-glactosamine hydrochloride-induced acute liver injury of rats.

Method: Randomly divide male SD rats into six groups according to their weights: a normal control group, a model control group, a low-dose dehydroapocavidine group, a medium-dose dehydroapocavidine group, a high-dose dehydroapocavidine group, and a positive control group, wherein each group contains ten rats. Feed the low-, medium- and high-dose groups with dehydroapocavidine in a gastric irrigation (ig) method respectively by doses of 20, 60, and 120 mg/kg. Feed the positive control group with diammonium glycyrrhetate in a gastric irrigation method by a dose of 75 mg/kg. Feed the normal control group and the model control group with distilled water in a gastric irrigation method by a dose of 5 ml/kg/d for seven days. On the sixth day after feeding dehydroapocavidine, inject 10% D-glactosamine hydrochloride solution into the abdominal cavities (ip) of the model group and the dehydroapocavidine-fed groups by a dose of 500 mg/kg. Feed normal saline into the abdominal cavities of the normal control group. 48 hours after ip feeding D-glactosamine hydrochloride, fast the rats overnight. Then, measure the activities of ALT, AST and ALP, and observe the histopathological variations of all the groups.

Result: The activities of ALT, AST and ALP of the model control group are obviously higher than that of the normal control group (P<0.01). All of the low-, medium- and high-dose dehydroapocavidine feedings can obviously lower the activities of ALT, AST and ALP of rats having the D-glactosamine hydrochloride-induced acute liver injury. Dehydroapocavidine has almost the same protection effect as diammonium glycyrrhetate used in the positive control group. The dose of dehydroapocavidine correlates with the effect of reducing the activities of ALT, AST and ALP. The histopathological examination shows that the liver focal hydropic degeneration and vacuolar degeneration of the dehydroapocavidine-fed groups is less serious than the model control group, and that the quantities and extends of the abovementioned degenerations are dependent on the dose of dehydroapocavidine. In fact, necrotic pathological change is hard to find in the livers of the high-dose group, and diffuse or local liver cell swelling is the primary pathological change in the high-dose group.

Conclusion: Ig dehydroapocavidine feeding can reduce extend of ALT, AST and ALP activity rising caused by the D-glactosamine hydrochloride-induced acute liver injury and decrease the seriousness of liver pathological change of the D-glactosamine hydrochloride-induced acute liver injury. Therefore, dehydroapocavidine can protect rats from the D-glactosamine hydrochloride-induced acute liver injury in a certain extent.

7. Dehydroapocavidine Protection Effect on the Phenylisothiocyanate-Induced Acute Liver Injury of Rats

Abstract

Objective: observing the dehydroapocavidine protection effect on the phenylisothiocyanate-induced acute liver injury of rats.

Method: Randomly divide male SD rats into six groups according to their weights: a normal control group, a model control group, a low-dose dehydroapocavidine group, a medium-dose dehydroapocavidine group, a high-dose dehydroapocavidine group, and a positive control group, wherein each group contains ten rats. Feed the low-, medium- and high-dose groups with dehydroapocavidine in a gastric irrigation (ig) method respectively by doses of 20, 60, and 120 mg/kg. Feed the positive control group with diammonium glycyrrhetate in a gastric irrigation method by a dose of 75 mg/kg. Feed the normal control group and the model control group with distilled water in a gastric irrigation method by a dose of 5 ml/kg/d for seven days. On the sixth day after feeding dehydroapocavidine, ig-feed every group with 1.1% (w/v) olive oil solution of phenylisothiocyanate by an amount of 10 ml/kg (a dose of 110 mg/kg) except the normal control group. The normal control group is only ig-fed with olive oil. 48 hours after feeding phenylisothiocyanate, fast the rats overnight. Then, measure the concentrations of ALT, AST, ALP, Tbil and Dbil, and observe the histopathological variations of all the groups.

Result: The concentrations of ALT, AST, ALP, Tbil and Dbil of the model control group are obviously higher than that of the normal control group (P<0.01). All of the low-, medium- and high-dose dehydroapocavidine feedings can obviously lower the activities of ALT, AST and ALP of rats having the phenylisothiocyanate-induced acute liver injury. Dehydroapocavidine has almost the same protection effect as diammonium glycyrrhetate used in the positive control group. The dose of dehydroapocavidine correlates with the effect of reducing the concentrations of ALT, AST, ALP, Tbil and Dbil. The histopathological examination shows that preventive ig feeding dehydroapocavidine can relieve the phenylisothiocyanate-induced acute liver injury in a certain extent, and that liver injury of the dehydroapocavidine-fed groups is less serious than the model control group, and that the quantities and extends of the degenerations are dependent on the dose of dehydroapocavidine. In fact, most of the livers of the high-dose group are in a normal state.

Conclusion: Ig dehydroapocavidine feeding can reduce extend of ALT, AST, ALP, Tbil and Dbil activity rising caused by the phenylisothiocyanate-induced acute liver injury. Therefore, dehydroapocavidine can protect rats from the phenylisothiocyanate-induced acute liver injury in a certain extent.

8. Dehydroapocavidine Protection Effect on the Carbon Tetrachloride-Induced Acute Liver Injury of Rats

Abstract

Objective: observing the dehydroapocavidine protection effect on the carbon tetrachloride-induced acute liver injury of rats.

Method: Randomly divide male SD rats into six groups according to their weights: a normal control group, a model control group, a low-dose dehydroapocavidine group, a medium-dose dehydroapocavidine group, a high-dose dehydroapocavidine group, and a positive control group, wherein each group contains ten rats. Feed the low-, medium- and high-dose groups with dehydroapocavidine in a gastric irrigation (ig) method respectively by doses of 20, 60, and 120 mg/kg. Feed the positive control group with diammonium glycyrrhetate in a gastric irrigation method by a dose of 75 mg/kg. Feed the normal control group and the model control group with distilled water in a gastric irrigation method by a dose of 5 ml/kg/d for seven days. On the sixth day after feeding dehydroapocavidine, subcutaneously inject (sc) 25% (V/V) carbon tetrachloride solution into the backs of the model control group and the dehydroapocavidine-fed groups by a dose of 2 ml/kg to induce acute liver injury. Subcutaneously inject olive oil into the backs of the normal control group. 48 hours after injecting carbon tetrachloride, fast the rats overnight. Then, measure the activities of ALT, AST and ALP, and observe the histopathological variations of all the groups.

Result: The activities of ALT, AST and ALP of the model control group are obviously higher than that of the normal control group (P<0.01). All of the preventive low-, medium- and high-dose dehydroapocavidine feedings can obviously lower the activities of ALT, AST and ALP of rats having the carbon tetrachloride-induced acute liver injury. Dehydroapocavidine has almost the same protection effect as diammonium glycyrrhetate used in the positive control group. The dose of dehydroapocavidine correlates with the effect of reducing the activities of ALT, AST and ALP. The histopathological examination shows that the hepatocellular focal ballooning degeneration and vacuolar degeneration of the dehydroapocavidine-fed groups is less serious than the model control group, and that the quantity of the early necrotic liver cells, the range and extend of degeneration of the dispersively distributed focuses decrease with the increasing dose of dehydroapocavidine. In fact, necrotic pathological change is hard to find in the livers of the high-dose group.

Conclusion: Ig dehydroapocavidine feeding can reduce extend of ALT, AST and ALP activity rising caused by the carbon tetrachloride-induced acute liver injury. Therefore, dehydroapocavidine can protect rats from the carbon tetrachloride-induced acute liver injury in a certain extent.

9. Dehydroapocavidine Protection Effect on the Carbon Tetrachloride-Induced Chronic Liver Injury of Rats

Abstract

Objective: observing the dehydroapocavidine protection effect on the carbon tetrachloride-induced chronic liver injury of rats.

Method: Randomly divide male SD rats into six groups according to their weights: a normal control group, a model control group, a low-dose dehydroapocavidine group, a medium-dose dehydroapocavidine group, a high-dose dehydroapocavidine group, and a positive control group, wherein each group contains twenty rats. Subcutaneously inject 25% (V/V) olive oil solution of carbon tetrachloride into the rats of every group by dose of 2 ml/kg twice a week for twelve weeks except the normal control group. From the fifth week, feed the low-, medium- and high-dose groups with dehydroapocavidine in a gastric irrigation (ig) method respectively by doses of 20, 40, and 80 mg/kg once per day for eight weeks. Feed the model control group with distilled water in a gastric irrigation method. Feed the positive control group with diammonium glycyrrhetate in a gastric irrigation method. Feed the normal control group with olive oil by a dose of 2 ml/kg twice a week for twelve weeks. From the fifth week, feed the normal control group with distilled water in a gastric irrigation (ig) method once per day for eight weeks. Twenty four hours after the last cycle of dehydroapocavidine feeding, paralyze ten rats of each group to take blood samples from aorta abdominalis to measure the concentrations of ALT, AST, ALP, TP, Alb and HYP. Obtain the absolute weights and relative weights of the livers, and observe the histopathological changes of the livers of every group.

Result: The concentrations of ALT, AST, ALP and HYP of the model control group are obviously higher than that of the normal control group (P<0.01). All of the preventive low-, medium- and high-dose dehydroapocavidine feedings can obviously lower the concentrations of ALT, AST, ALP and HYP of rats having the carbon tetrachloride-induced chronic liver injury. The dose of dehydroapocavidine correlates with the effect of reducing the concentrations of ALT, AST, ALP and HYP. The medium- and high-dose dehydroapocavidine can decrease the count in liver histopathology. Every dose of dehydroapocavidine can inhibit liver fibrosis and reduce liver degeneration. Therefore, dehydroapocavidine can prevent rats from the carbon tetrachloride-induced chronic liver injury.

Conclusion: Subcutaneous carbon tetrachloride injection induces chronic liver injury of rats. Dehydroapocavidine can reduce ALT, AST, ALP and HYP concentration rising caused by the carbon tetrachloride-induced chronic liver injury and prevent from liver fibrosis, wherein the effect of dehydroapocavidine correlates with the dose thereof. Therefore, dehydroapocavidine can protect rats from the carbon tetrachloride-induced chronic liver injury in a certain extent.

10. Influence of Dehydroapocavidine on the Bile Secretion of Normal SD Rats

Objective: observing the influence of dehydroapocavidine on the bile secretion of normal SD rats.

Method: Respectively feed single doses of dehydroapocavidine by 20, 60, 120 mg/kg into the gastric cavities of SD rats. Observe the influence of dehydroapocavidine on the bile secretion of normal SD rats with a bile drainage method.

Result: All of the low dose, medium dose and high dose of dehydrocavidine can obviously increase the bile secretion of the SD rats. Within one hour of dehydroapocavidine feeding, the total bile secretions of the three dehydrocavidine-injection groups are respectively 37.4%, 49.5% and 63.7% greater than that of the NS control group—0.91±0.05 ml with P<0.01 for all of them. Within two hour of dehydroapocavidine feeding, the total bile secretions of the three dehydrocavidine-injection groups are respectively 36.6%, 47.7% and 63.4% greater than that of the NS control group—1.72±0.08 ml with P<0.01 for all of them.

Conclusion: the gastric feeding of dehydroapocavidine can increase the bile secretion of normal SD rats. Therefore, dehydroapocavidine can promote the function of gall in a certain extend.

11. Influence of Dehydroapocavidine on the Immunological Function of Mice

Objective: Observing the influence of oral dehydroapocavidine feeding on the nonspecific immunological function of normal mice and the immunological function of body fluid.

Method: The HC50 values of mouse serum hemolysin are used to observe the influence of dehydroapocavidine on the immunological function of mouse body fluid. The clearance rates of charcoal particles are used to observe the influence of dehydroapocavidine on the nonspecific immunological function of mice.

Result: Inject dehydroapocavidine into the gastric cavities of BABL/C mice by doses of 30, 90 and 180 mg/kg/d for five days. The HC50 values of mouse serum hemolysin of the low-dose, medium-dose and high-dose dehydroapocavidine-feeding groups are respectively 20.0%, 20.7% and 22.1% higher than that of the NS control group—54.4±11.4 with P<0.05 for all of them. Inject dehydroapocavidine into the gastric cavities of Kunming mice by doses of 30, 90 and 180 mg/kg/d for five days. The clearance indexes K of the low-dose, medium-dose and high-dose dehydroapocavidine-feeding groups are respectively 7.1%, 29.5% and 33.1% higher than that of the NS control group—0.0372±0.0095 with P<0.05 (for the medium-dose and high-dose groups). The phagocytosis indexes a of the low-dose, medium-dose and high-dose dehydrocavidine-feeding groups are respectively 3.0%, 15.2% and 21.3% higher than that of the NS control group—5.34±0.84 with P<0.05 (for the medium-dose and high-dose groups).

Conclusion: Oral dehydroapocavidine feeding favors the generation of SRBC antibody in mice and promotes the immunological function of mouse body fluid. Oral feeding of a higher dose of dehydroapocavidine can increase the clearance rate of charcoal particles for mice and promote the phagocytosis function of monocytic macrophages. Therefore, dehydroapocavidine can enhance the nonspecific immunological function of mice.

What is claimed is:

1. A method of preparing dehydrocavidine-dehydroapocavidine composition and their respective individual compounds comprises:
isolating and purifying the quaternary ammonium alkaloid species from a medicinal plant of Yan Huang Lian (Corydalis saxicola Bounting) through solvent extraction, water-phase organic extraction, crystallization and recrystallization; and
using drying methods to prepare and obtain dehydrocavidine-dehydroapocavine composition, the composition or the crude extracts obtained from said steps can be separated by chromatography to obtain individual compounds of dehydrocavidine and dehydroapocavidine.

2. The method as claimed in claim 1, wherein the solvents of said solvent extraction can be water, acidic water, methanol, ethanol, propanol, butanol and ethyl acetate, or a mixture of these solvents.

3. The method as claimed in claim 2, wherein the content of dehydrocavidine and dehydroapocavidine within the composition is in the range of 5% to 99.5% (w/w).

4. The method as claimed in claim 1, wherein said preparation method further comprises, in said solvent extraction, employing ultrasonic extracting, percolation extracting or reflux extracting.

5. The method as claimed in claim 4, wherein the content of dehydrocavidine and dehydroapocavidine within the composition is in the range of 5% to 99.5% (w/w).

6. The method as claimed in claim 1, wherein the extracts obtained in said water-phase extraction are dispersed in water, defatted with petroleum ether and extracted with appropriate organic solvents in order to remove the non-quaternary ammonium alkaloid species.

7. The method as claimed in claim 6, wherein the organic solvents used in said water-phase organic extraction can be chloroform, dichloromethane, ether, acetate ether, ethyl acetate, or butanol.

8. The method as claimed in claim 7, wherein the content of dehydrocavidine and dehydroapocavidine within the composition is in the range of 5% to 99.5% (w/w).

9. The method as claimed in claim 6, wherein the content of dehydrocavidine and dehydroapocavidine within the composition is in the range of 5% to 99.5% (w/w).

10. The method as claimed in claim 1, wherein the extracts obtained in said water-phase organic extraction are dispersed in water; and the non-quaternary ammonium alkaloid species can be removed directly with appropriate organic solvents.

11. The method as claimed in claim 10, wherein the organic solvents used in said water-phase organic extraction can be chloroform, dichloromethane, ether, acetate ether, ethyl acetate, or butanol.

12. The method as claimed in claim 11, wherein the content of dehydrocavidine and dehydroapocavidine within the composition is in the range of 5% to 99.5% (w/w).

13. The method as claimed in claim 10, wherein the content of dehydrocavidine and dehydroapocavidine within the composition is in the range of 5% to 99.5% (w/w).

14. The method as claimed in claim 1, wherein in the method of crystallization, the solvents can be water, methanol, ethanol, butanol, acetone, or their mixture.

15. The method as claimed in claim 14, wherein the content of dehydrocavidine and dehydroapocavidine within the composition is in the range of 5% to 99.5% (w/w).

16. The method as claimed in claim 1, wherein in said methods of recrystallization, the solvents used can be methanol, ethanol, water, acidic water, acidic methanol, acidic ethanol or their mixture.

17. The method as claimed in claim 16, wherein the content of dehydrocavidine and dehydroapocavidine within the composition is in the range of 5% to 99.5% (w/w).

18. The method as claimed in claim 1, wherein in said method of recrystallization, the acid used can be hydrochloric acid, sulfuric acid, phosphoric acid, nitric acid, perchloric acid, succinic acid, oxalic acid, acetic acid, formic acid, or their mixture.

19. The method as claimed in claim 18, wherein the content of dehydrocavidine and dehydroapocavidine within the composition is in the range of 5% to 99.5% (w/w).

20. The method as claimed in claim 1, wherein the drying method can be decompression drying, spray drying or freeze drying or their combination.

21. The method as claimed in claim 20, wherein the content of dehydrocavidine and dehydroapocavidine within the composition is in the range of 5% to 99.5% (w/w).

22. The method as claimed in claim 1, wherein filling materials for chromatography can be silica gel, aluminum oxide, polyamide, sephadex gel, or their mixture.

23. The method as claimed in claim 22, wherein the content of dehydrocavidine and dehydroapocavidine within the composition is in the range of 5% to 99.5% (w/w).

24. The method as claimed in claim 1, wherein the chromatography can be column or thin layer, or their combination.

25. The method as claimed in claim 24, wherein the content of dehydrocavidine and dehydroapocavidine within the composition is in the range of 5% to 99.5% (w/w).

26. The applications of the dehydrocavidine-dehydroapocavidine composition in pharmaceuticals treating acute and chronic virus hepatitis, liver injury, influenza, tumors, AIDS, and arrhythmia; and the applications of individual compounds of dehydrocavidine and dehydroapocavidine in pharmaceuticals treating acute and chronic virus hepatitis, liver injury, influenza, tumors, AIDS and arrhythmia as well.

27. The pharmaceutical compositions for treating acute and chronic viral hepatitis, liver injury, influenza, tumors, AIDS and arrhythmia, comprise the therapeutically effective quantity of dehydrocavidine-dehydroapocavidine composition and individual compounds of dehydrocavidine and dehydroapocavidine, and the pharmaceutically acceptable excipients.
